(19) United States
(12) Patent Application Publication
(10) Pub. No.: US 2006/0193895 A1
(43) Pub. Date: Aug. 31, 2006

(54) Additive for food and beverage, pharmaceutical composition, GLUT4 translocator, and method for translocating GLUT4

(75) Inventor: Toshihiro Miura, Suzuka-shi (JP)

Correspondence Address: MORGAN LEWIS & BOCKIUS LLP, 1111 PENNSYLVANIA AVENUE NW, WASHINGTON, DC, 20004, US

(73) Assignee: Use-Techno Corporation

(21) Appl. No.: 11/066,331

(22) Filed: Feb. 25, 2005

(51) Int. Cl. A61K 47/00; A61K 31/20

(52) U.S. Cl. 424/439; 514/559

(57) ABSTRACT

An additive for food and beverage, comprising a compound represented by the general formula (1) or a salt thereof: 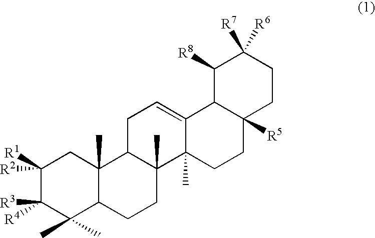
wherein R¹ to R⁴ are each a glycoside group, a hydroxyl group, or a hydrogen atom, R⁵ is a carboxyl group and R⁶ to R⁸ are each a methyl group or a hydrogen atom.

### BACKGROUND OF THE INVENTION

1. Field of the Invention

The present invention relates to an additive for food and beverage, a food and a beverage containing the additive, a pharmaceutical composition for treating and preventing diabetes or hyperglycemia, a GLUT4 translocator for translocating glucose transporter type 4 (GLUT4) onto cell membrane, and a method for translocating glucose transporter type 4 (GLUT4) onto cell membrane.

2. Related Background of the Invention

Diabetes mellitus is a group of metabolic disorders characterized by chronic hyperglycemia as a result of insufficient insulin action. Diabetes is divided broadly into two major categories: Type I diabetes caused by the destruction and disappearance of Langerhans' β cells in pancreas for synthesizing and secreting insulin; and Type II diabetes involved with environmental factors such as hyperphagia, obesity and stress, and genetic factors such as hyposecretion of insulin.

Although insulin is used to treat diabetes, the oral defective absorption keeps it to be administered only by injection. In this regard, oral hypoglycemic agents such as sulfonyl urea, biguanide, α-glucosidase inhibitors, thiazolidine derivative and phenylalanine derivative, that can be administered more simply than injection of insulin, have been developed specifically for Type II diabetes.(See “Guideline for curing diabetes: 2002-2003”, 1st print of 1st edition, May, 9, 2002, edited by Japan Diabetes Society, published by Bunkodo Co., Ltd.)

### SUMMARY OF THE INVENTION

However, the aforementioned conventional oral hypoglycemic agents easily induce hypoglycemia which causes finger tremor, paling, decreased level of consciousness, convulsion, coma and the like. Consequently, glucose or a glucose-containing beverage and the like must be always carried to cope with the symptoms.

In view of this point, an object of the present invention is to provide a material applicable for treating or preventing diabetes or hyperglycemia, which material can be orally administrated and hardly induce hypoglycemia after administration.

The inventors of the present invention carried out an extensive study and found that corosolic acid and its analogous compound can be used as the additive for food and beverage or in a pharmaceutical composition to achieve the above object, and thus completed the present invention.

That is, the present invention provides an additive for food and beverage, comprising a compound represented by the general formula (1) or a salt thereof: 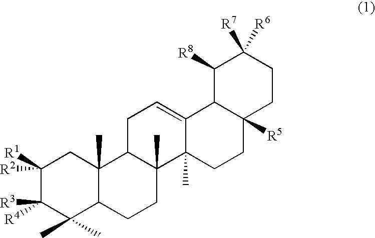
wherein R¹, R², R³ and R⁴ independently represent a glycoside group consisting of 1 to 10 monosaccharide units, a hydroxyl group or a hydrogen atom, R⁵ represents a carboxyl group, a methyl group, a methylol group, a formyl group or a group represented by —COR¹⁰, R⁶, R⁷ and R⁸ independently represent a methyl group or a hydrogen atom, wherein R¹⁰ is a glycoside group consisting of 1 to 10 monosaccharide units, and two of R⁶, R⁷ and R⁸ are each methyl group.

The present invention also provides a pharmaceutical composition for treating or preventing diabetes or hyperglycemia, comprising a compound represented by the above general formula (1) or a salt thereof as an active ingredient. The compound represented by the general formula (1) or a salt thereof for the additive for food and beverage or in the pharmaceutical composition is preferably derived from Banaba leaves.

The additive for food and beverage and the pharmaceutical composition described above can be orally administrated, and can be administrated to a diabetic patient without the trouble of hypoglycemia induced. Generally, blood sugar level rapidly increases immediately after meal, followed by intrinsic insulin secretion which plays a role for suppressing the present increase in blood sugar level. Since, however, insulin is secreted a little after the rapid increase in blood sugar level, the secretion fails to respond quickly to the rapid increase in blood sugar level after meal. Such phenomenon occurs not only in the diabetic patients but also in normal persons. Such time lag prevents the blood sugar level from rapid increasing inside blood vessels, which causes increase of blood cholesterol level, circulatory diseases, stress on the kidney and optic nerve disorders.

The additive for food and beverage and the pharmaceutical composition have an excellent effect to solve the problem caused by the aforementioned time lag. Intake of sugar into cells is carried out through the translocation of the glucose transporter type 4 (GLUT4) onto cell membrane. The inventors of the present invention found that a compound represented by the general formula (1) or a salt thereof, which is a main ingredient of the aforementioned additive for food and beverage and the pharmaceutical composition, enhances the translocation of GLUT4 onto cell membrane. Consequently, a composition containing a compound represented by the general formula (1) or a salt thereof can be administered orally before meal to translocate GLUT4 onto cell membrane in advance, which can prevent blood sugar level from abrupt increase after meal to solve the above trouble caused by the aforementioned time lag.

That is, the present invention provides a GLUT4 translocator which has a compound represented by the above general formula (1) or a salt thereof as an active ingredient in order to translocate glucose transporter type 4 (GLUT4) onto cell membrane, and a method for translocating the glucose transporter type 4 (GLUT4) onto cell membrane by orally administrating a composition containing a compound represented by the general formula (1) or a salt thereof before meal.

In the aforementioned additive for food and beverage, pharmaceutical composition, GLUT4 translocator, and method for tranlocating GLUT4, the combination of R¹, R², R³, R⁴, R⁵, R⁶, R⁷ and R⁸ in the general formula (1) is preferably: (a) R¹ being a hydrogen atom, R² being a hydroxyl group, R³ being a hydroxyl group, R⁴ being a hydrogen atom, R⁵ being a carboxyl group, R⁶ being a methyl group, R⁷ being a hydrogen atom and R⁸ being a methyl group; (b) R¹ being a hydrogen atom, R² being a hydroxyl group, R³ being a hydroxyl group, R⁴ being a hydrogen atom, R⁵ being a carboxyl group, R⁶ being a methyl group, R⁷ being a methyl group and R⁸ being a hydrogen atom; (c) R¹ being a hydrogen atom, R² being a hydrogen atom, R³ being a hydroxyl group, R⁴ being a hydrogen atom, R⁵ being a carboxyl group, R⁶ being a methyl group, R⁷ being a hydrogen atom, and R⁸ being a methyl group; or (d) R¹ being a hydrogen atom, R² being a hydrogen atom, R³ being a hydroxyl group, R⁴ being a hydrogen atom, R⁵ being a carboxyl group, R⁶ being a methyl group, R⁷ being a methyl group and R⁸ being a hydrogen atom.

In the aforementioned additive for food and beverage, pharmaceutical composition, GLUT4 translocator, and method for translocating GLUT4, a plurality of compounds in accordance with the general formula (1) may be used in combination. It is preferable to apply a composition comprising at least one compound or salt thereof selected from the group consisting of: maslinic acid represented by the general formula (1) in which R¹ is a hydrogen atom, R² is a hydroxyl group, R³ is a hydroxyl group, R⁴ is a hydrogen atom, R⁵ is a carboxyl group, R⁶ is a methyl group, R⁷ is a methyl group and R⁸ is a hydrogen atom; ursolic acid represented by the general formula (1) in which R¹ is a hydrogen atom, R² is a hydrogen atom, R³ is a hydroxyl group, R⁴ is a hydrogen atom, R⁵ is a carboxyl group, R⁶ is a methyl group, R⁷ is a hydrogen atom and R⁸ is a methyl group; and oleanolic acid represented by the general formula (1) in which R¹ is a hydrogen atom, R² is a hydrogen atom, R³ is a hydroxyl group, R⁴ is a hydrogen atom, R⁵ is a carboxyl group, R⁶ is a methyl group, R⁷ is a methyl group and R⁸ is a hydrogen atom; and corosolic acid represented by the general formula (1) or a salt thereof in which R¹ is a hydrogen atom, R² is a hydroxyl group, R³ is a hydroxyl group, R⁴ is a hydrogen atom, R⁵ is a carboxyl group, R⁶ is a methyl group, R⁷ is a hydrogen atom and R⁸ is a methyl group.

The compound represented by the above general formula (1) or a salt thereof is preferably derived from Banaba leaves. By adding the above composition for food and beverage, there can be provided a food or a beverage which can be administrated orally before meal to prevent blood sugar level from abrupt increasing.

### BRIEF DESCRIPTION OF THE DRAWINGS

FIG. 1 is a graph showing the change in blood sugar level with time in KK-Ay mice with corosolic acid or a comparative substance (for control) administrated.

FIG. 2 is a graph showing the serum insulin values in KK-Ay mice with corosolic acid or a comparative substance (for control) administrated.

FIG. 3 is a graph showing the relative quantities (%) of GLUT4 in the PM fraction for KK-Ay mice with corosolic acid or a comparative substance (for control) administrated.

FIG. 4 is a graph showing the relative quantities (%) of GLUT4 in the LDM fraction for KK-Ay mice with corosolic acid or a comparative substance (for control) administrated.

FIG. 5 shows the result of western blotting test.

FIG. 6 is a graph showing the change in blood sugar level with time in ddY mice with corosolic acid or a comparative substance (for control) administrated.

### DESCRIPTION OF THE PREFERRED EMBODIMENTS

Regarding the compound represented by the general formula (1) and a salt thereof according to the present invention, preferred embodiments are described below.

In a compound represented by the general formula (1), R¹, R², R³ and R⁴ may be a glycoside group consisting of 1 to 10 monosaccharide units, and R¹⁰ in a —COR¹⁰ group for R⁵ may also be a glycoside group consisting of 1 to 10 monosaccharide units. That is, the compound represented by the general formula (1) may be a glycoside. The glycoside group in this case has preferably 1 to 6 monosaccharide units (without limitation in the number of carbons composing the monosaccharide unit), and more preferably 1 to 3 units.

Specifically preferable compounds represented by the general formula (1) are corosolic acid and an analogous compound thereof. That is, preferable compound includes corosolic acid (represented by the formula (1a) in which R¹ is a hydrogen atom, R² is a hydroxyl group, R³ is a hydroxyl group, R⁴ is a hydrogen atom, R⁵ is a carboxyl group, R⁶ is a methyl group, R⁷ is a hydrogen atom and R⁸ is a methyl group), maslinic acid (represented by the formula (1b) in which R¹ is a hydrogen atom, R² is a hydroxyl group, R³ is a hydroxyl group, R⁴ is a hydrogen atom, R⁵ is a carboxyl group, R⁶ is a methyl group, R⁷ is a methyl group and R⁸ is a hydrogen atom), ursolic acid (represented by the formula (1c) in which R¹ is a hydrogen atom, R² is a hydrogen atom, R³ is a hydroxyl group, R⁴ is a hydrogen atom, R⁵ is a carboxyl group, R⁶ is a methyl group, R⁷ is a hydrogen atom and R⁸ is a methyl group) and oleanolic acid (represented by the formula (1d) in which R¹ is a hydrogen atom, R² is a hydrogen atom, R³ is a hydroxyl group, R⁴ is a hydrogen atom, R⁵ is a carboxyl group, R⁶ is a methyl group, R⁷ is a methyl group and R⁸ is a hydrogen atom).

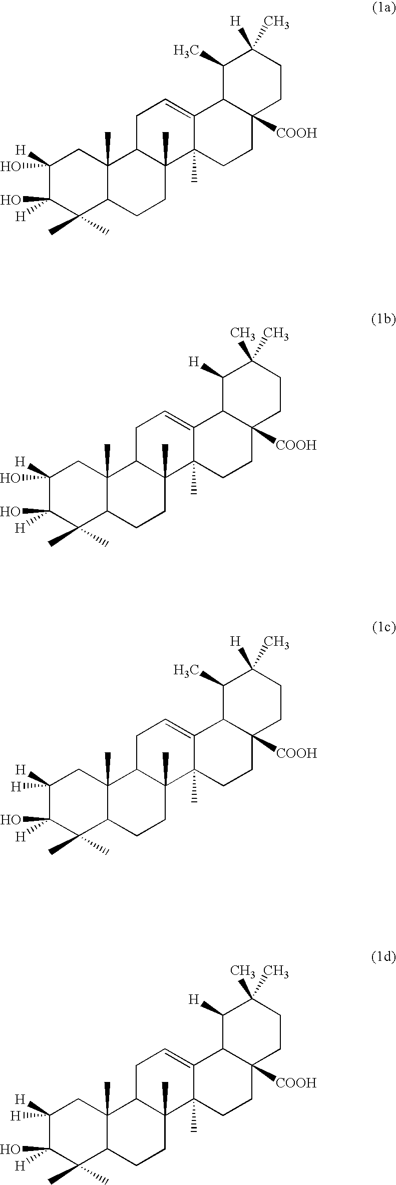

Among these compounds, corosolic acid is most preferable, and corosolic acid may be combined with at least one of maslinic acid, ursolic acid and oleanolic acid (preferably at least one of maslinic acid and ursolic acid). For combining them, it is preferable to combine 0.1 to 10 parts by weight, preferably 1 to 10 parts by weight, and more preferably 5 to 10 parts by weight of at least one of maslinic acid, ursolic acid, and oleanolic acid with 100 parts by weight of corosolic acid.

The compound represented by the general formula (1) may form a salt thereof. Examples of such salt include metallic salts such as sodium salt, potassium salt and magnesium salt.

The compound represented by the general formula (1) is preferably derived from Banaba leaves (Lagerstroemia Speciosa, Linn. or Pers.). In particular, corosolic acid, maslinic acid, ursolic acid and oleanolic acid are preferably derived from Banaba leaves by extraction. In order to obtain a compound represented by the general formula (1) from Banaba leaves, Banaba leaves is subjected to extraction using alcohol or the like to obtain Banaba extract (which is further concentrated to get Banaba extract concentrate if needed), which is then purified.

Raw Banaba leaves or dried Banaba leaves are preferable for extraction. Applicable method for drying raw Banaba leaves includes natural drying, air drying and forced drying. To prevent growth of microorganisms and to assure preservation stability, the leaves are preferably dried by toasting to have a water content of 20% by weight or less, and more preferably 10% by weight or less. The dried Banaba leaves may be directly extracted or may be crushed or cut to fine chips before the extraction.

Banaba leaves thus prepared are subjected to extraction using a solvent such as hot water or alcohol such as methanol and ethanol to obtain Banaba extract. In this case, it is preferable to adopt an extraction condition that allows the corosolic acid and the analogous compounds thereof (such as maslinic acid, ursolic acid, and oleanolic acid) to exist in the extract at a certain rate. Examples of such extraction method include the following methods 1 to 3.

Method 1: To the dried and crushed Banaba leaves (material), 5 to 20 fold by weight, preferably 8 to 10 fold by weight, of ethanol or aqueous ethanol (50 to 80% by weight of ethanol) relative to the material is added. The mixture is heated under reflux at a temperature of normal temperature to 90° C., preferably about 50 to 85° C., for 30 minutes to 2 hours. This extraction is repeated twice or thrice.

Method 2: To the dried and crushed Banaba leaves, 3 to 20 fold by weight of ethanol or aqueous ethanol (50 to 90% by weight of ethanol) is added. The mixture is heated under reflux to extract under the same condition as in Method 1. Extraction is preferably executed at a temperature of normal temperature to 65° C. for 30 minutes to 2 hours. This extraction may be executed not only once, but also twice or more.

Method 3: To the dried and crushed Banaba leaves, 3 to 20 fold by weight of hot water is added. The mixture is heated under reflux to extract at a temperature of 50 to 90° C., preferably 60 to 85° C., for 30 minutes to 2 hours.

The above Methods 1 to 3 for Banaba leaves extraction may be combined with each other. For example, Method 1 and Method 2 may be combined together. Among these three methods, Method 1 and Method 2 are preferred, and Method 1 is particularly preferred.

Banaba extract thus prepared is purified to obtain the compound represented by the general formula (1). A known method is applicable for the purification. For the case of purifying corosolic acid, for example, the following method is preferably employed.

The Banaba extract is suspended in water, which is then distributed in ether, hexane or the like to remove low polar components. The water layer is eluted by water, methanol, and acetone in this order using DIA-ION HP-20 column chromatography or the like. The methanol elution fraction which contains corosolic acid is further subjected to silica gel chromatography and high performance liquid chromatography for separation and purification to isolate corosolic acid. The extract (particularly in the case of a large amount of the extract) can be easily purified by removing the low polar components by ether, hexane or the like, and then separating by DIA-ION HP-20 column chromatography, but these steps are not necessarily essential. It is possible that the extract is directly separated by silica gel column chromatography, and then ultimately subjected to purification by high performance liquid chromatography.

The additive for food and beverage, the pharmaceutical composition, and the GLUT4 translocator according to the present invention contain a compound represented by the general formula (1) at an arbitrary content. To attain sure effect with a small amount, the additive for food and beverage, the pharmaceutical composition, and the GLUT4 translocator according to the present invention preferably each consist of the compound represented by the general formula (1). The compound represented by the general formula (1) may be used as a single species or in combination of a plurality of the species.

The additive for food and beverage, the pharmaceutical composition, and the GLUT4 translocator are applicable to human or non-human animals. The subjects to apply may be human or non-human animals with or without diabetes.

The additive for food and beverage, the pharmaceutical composition, and the GLUT4 translocator are preferably administrated orally before meal. The administration is preferably adjusted in timing for an individual so that glucose transporter type 4 (GLUT4) may have been translocated onto cell membrane at the meal. In the case of human, for instance, the administration is preferably done 0 minute to 2 hours before meal. A preferred dose is 0.1 to 1000 mg for 60 kg of subject body weight.

The additives for food and beverage may be added to beverages such as water, soft drink, fluid juice, milk and alcoholic beverage, and foods such as bread, noodle, rice, tofu, dairy products, soy sauce, miso and cake. The amount to add is preferably adjusted to give an amount of 0.1 to 1,000 mg for 60 kg of subject body weight.

### EXAMPLES

The present invention is described in more detail with respect to the following preferred examples. The scope of the present invention is not limited to the examples.

(Blood Sugar Level in KK-Ay Mouse with Administration of Corosolic Acid)

KK-Ay mice (CLEA JAPAN, INC.) which are the model animals for Type II diabetes were employed for test animals. These KK-Ay mice aged 6 to 10 weeks, having 300 mg/dL of blood sugar level. Corosolic acid which was extracted from Banaba leaves and purified was forcefully administrated to these KK-Ay mice using an injection syringe and an oral sound for mouse.

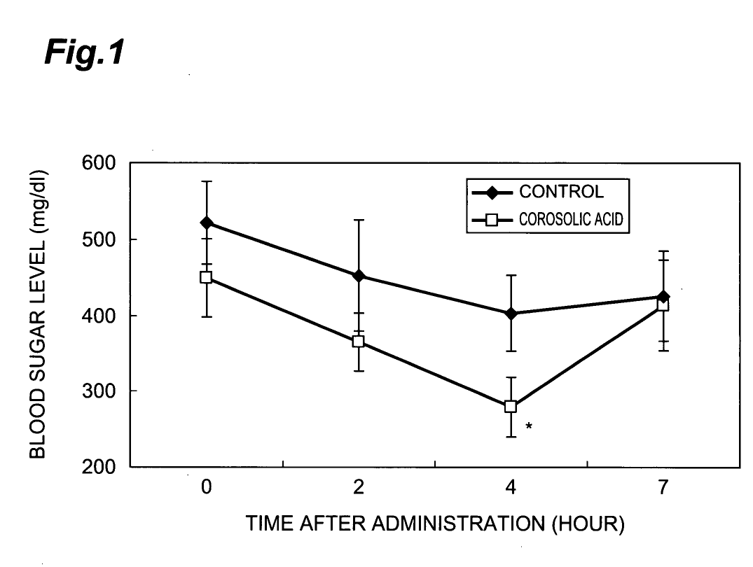

Blood samples were collected at 2, 4 and 7 hours after the administration using the orbital vein blood sampling method, and the blood sugar levels were determined by the glucose oxidase method. Blood sugar levels were measured on four KK-Ay mice. The measured values were used to calculate an average, which was evaluated by the Student's t-test. FIG. 1 shows the result (change in blood sugar level with time). FIG. 1 describes the blood sugar level data as average value± standard error. The p-value of 0.05 or below in the Student's t-test was rated to be significant (the significance is shown with * mark in the Figure: the same is applied hereinafter).

FIG. 1 reveals that in the KK-Ay mice of the group with corosolic acid administrated, the blood sugar level significantly decreases during 4 hours after the administration. It was also found that the blood sugar level after 4 hours from the administration of corosolic acid was about 280 mg/dL, suggesting that corosolic acid can be administrated to decrease the blood sugar level without hypoglycemia (a blood sugar level of 60 mg/dL or lower) induced.

(Serum Insulin Value in KK-Ay Mouse with Administration of Corosolic Acid)

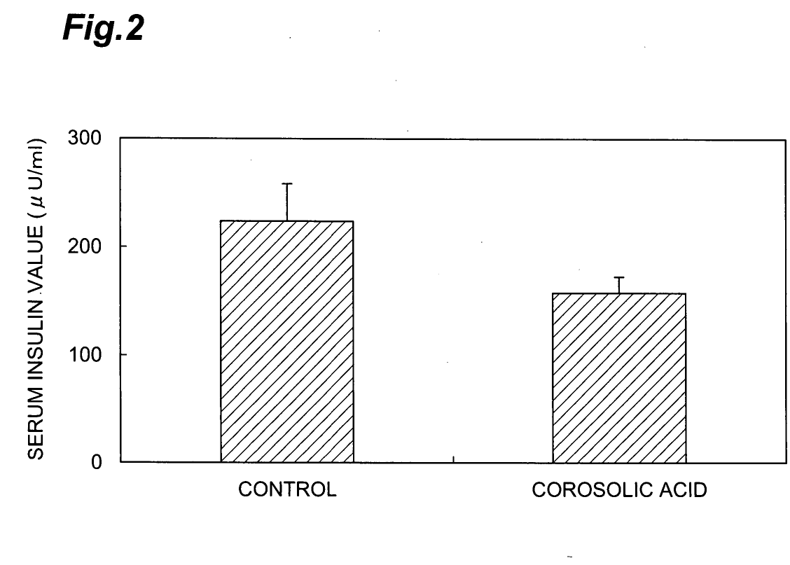

On the KK-Ay mouse groups with or without corosolic acid administrated in “Blood sugar level in KK-Ay mouse by administration of corosolic acid” described above, the serum insulin values were measured after 4 hours from the administration. The serum insulin values were measured by the enzyme 2-antibody method. The measured data were rated by the Student's t-test to determine whether or not they had significant differences according to the same criterion as above. FIG. 2 shows the result. The figure reveals that the group with corosolic acid administrated had no significant difference in serum insulin value from the group without corosolic acid administrated, suggesting that the serum insulin itself was not varied.

(Effect of Corosolic Acid on GLUT4 in Mouse Skeletal Muscle)

The intake of sugar into cells is conducted by a carrier protein in the transporting system on cell membrane, called glucose transporter (GLUT). Muscle has glucose transporter type 1 (GLUT1) and glucose transporter type 4 (GLUT4). The GLUT4 is known to migrate from a low density microsome fraction (LDM) to a plasma membrane (PM) to exhibit sugar-intake activity. This change in location within a cell is called GLUT4 translocation. It is speculated that significant increase of glucose in muscle and fat tissues is mainly due to the translocation of GLUT4. In view of this point, the effect of corosolic acid on GLUT4 was studied.

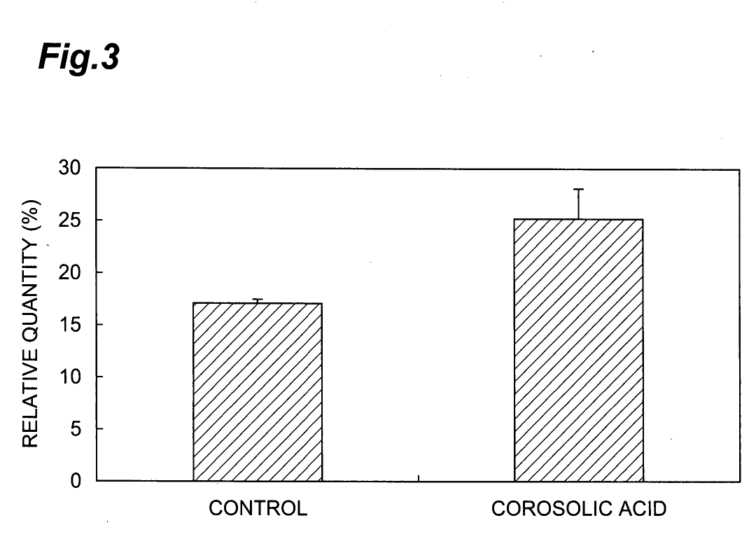

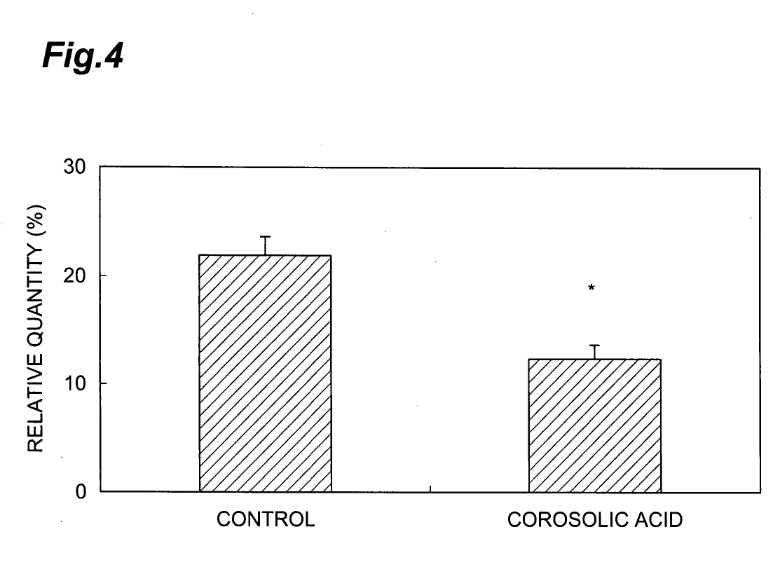

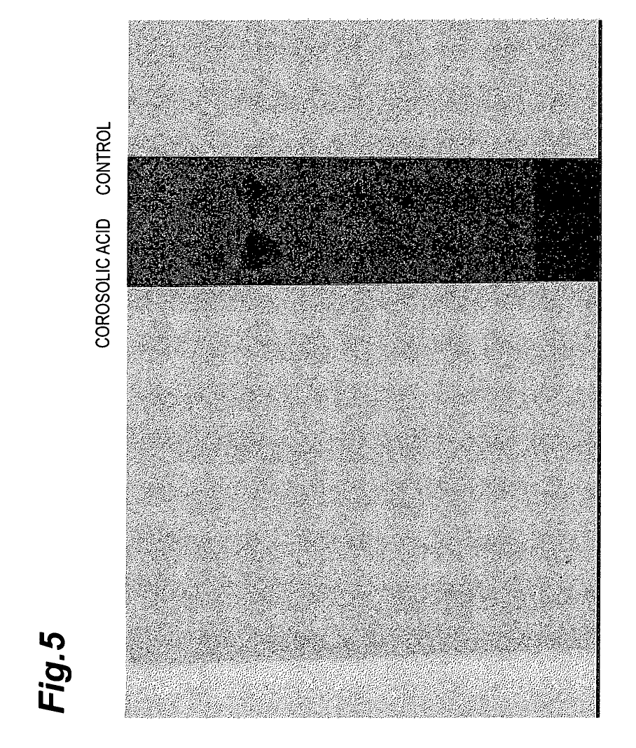

First, the low density microsome (LDM) fraction and the plasma membrane (PM) fraction were prepared using the sucrose concentration gradient in accordance with the method of Klip et al. (FEBS Lett., 224, 224-230 (1987), Biochem. Biophys. Res. Commum., 172, 728-736 (1990)). On the KK-Ay mouse groups with or without corosolic acid administrated, the relative quantities of GLUT4 in the PM fraction and the LDM fraction were determined after 4 hours from the administration using the western blotting analysis. FIG. 3 shows the relative quantity (%) of GLUT4 in the PM fraction, and FIG. 4 shows the relative quantity (%) of GLUT4 in the LDM fraction. As clearly shown in these figures, it was found that corosolic acid decreased the GLUT4 of low density microsome and increased it in the plasma membrane fraction, suggesting that the GLUT4 was translocated onto cell membrane. The phenomenon was also confirmed by the fact that the group with corosolic acid administrated was observed to give a denser spot in an experiment using the western blotting method (ECL method) (FIG. 5).

(Blood Sugar Level of ddY Mouse by Administration of Corosolic Acid)

Blood sugar level was determined by the same procedure as described in the above “Blood sugar level in KK-Ay mouse by administration of corosolic acid”, except that the ddY mice which are the normal mice (Japan SLC, Inc.) were employed instead of the KK-Ay mice which are the model animals for Type II diabetes.

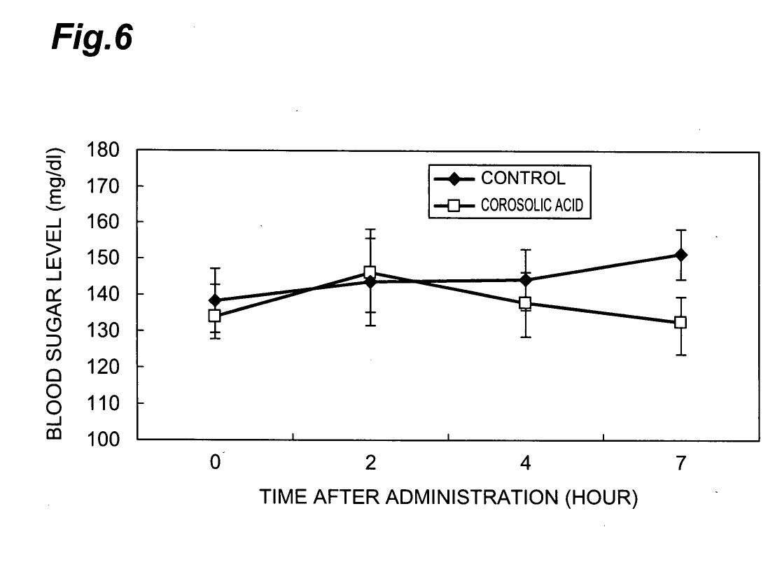

FIG. 6 shows the result (change in blood sugar level with time). No significant difference was observed in the data 2, 4, and 7 hours after the administration, suggesting that corosolic acid acts specifically on diabetes.

As described above, the present invention provides a material (compound) which can be orally administrated, hardly induces hypoglycemia after administration, and is applicable for treating or preventing diabetes or increase in blood sugar level. Such compound is a compound represented by the general formula (1) or a salt thereof, and is usable for an additive for food and beverage, a pharmaceutical composition for treating or preventing diabetes or hyperglycemia, and a GLUT4 translocator for translocating glucose transporter type 4 (GULT4) onto cell membrane. Addition of the additive for food and beverage can provide a food or beverage that has an effect of treating or preventing diabetes or hyperglycemia.

Furthermore, the compound represented by the above general formula (1) or a salt thereof is orally administrated before meal to allow translocation of the glucose transporter type 4 (GLUT4) onto cell membrane, exhibiting the effect of treating or preventing diabetes or hyperglycemia.

The compound represented by the above general formula (1) or a salt thereof can be obtained by extraction of Banaba leaves to provide an inexpensive preparation. In addition, the compound or a salt thereof, even if continuously administrated, gives no fear of a adverse effect and assures high safety. Furthermore, the compound or a salt thereof provides efficacy even if administrated by a small amount, and causes no adverse effect even if done by a large quantity.

What is claimed is:

1. An additive for food and beverage comprising a compound represented by the general formula (1) or a salt thereof: 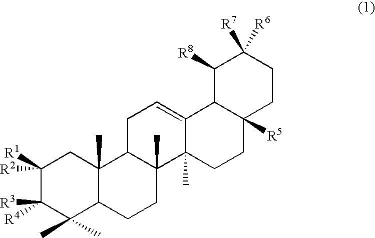
wherein
R¹, R², R³ and R⁴ independently represent a glycoside group consisting of 1 to 10 monosaccharide units, a hydroxyl group or a hydrogen atom,
R⁵ represents a carboxyl group, a methyl group, a methylol group, a formyl group or a group represented by —COR¹⁰,
R⁶, R⁷ and R⁸ independently represent a methyl group or a hydrogen atom,
wherein R¹⁰ is a glycoside group consisting of 1 to 10 monosaccharide units, and two of R⁶, R⁷ and R⁸ are each methyl group.

2. An additive for food and beverage according to claim 1,
wherein the combination of R¹, R², R³, R⁴, R⁵, R⁶, R⁷ and R⁸ in the general formula (1) is:
R¹ being a hydrogen atom, R2 being a hydroxyl group, R³ being a hydroxyl group, R⁴ being a hydrogen atom, R⁵ being a carboxyl group, R⁶ being a methyl group, R⁷ being a hydrogen atom and R⁸ being a methyl group;
R¹ being a hydrogen atom, R² being a hydroxyl group, R³ being a hydroxyl group, R⁴ being a hydrogen atom, R⁵ being a carboxyl group, R⁶ being a methyl group, R⁷ being a methyl group and R⁸ being a hydrogen atom;
R¹ being a hydrogen atom, R² being a hydrogen atom, R³ being a hydroxyl group, R⁴ being a hydrogen atom, R⁵ being a carboxyl group, R⁶ being a methyl group, R⁷ being a hydrogen atom and R⁸ being a methyl group; or
R¹ being a hydrogen atom, R² being a hydrogen atom, R³ being a hydroxyl group, R⁴ being a hydrogen atom, R⁵ being a carboxyl group, R⁶ being a methyl group, R⁷ being a methyl group and R⁸ being a hydrogen atom.

3. An additive for food and beverage according to claim 1,
wherein the combination of R¹, R², R³, R⁴, R⁵, R⁶, R⁷ and R⁸ in the general formula (1) is:
R¹ being a hydrogen atom, R² being a hydroxyl group, R³ being a hydroxyl group, R⁴ being a hydrogen atom, R⁵ being a carboxyl group, R⁶ being a methyl group, R⁷ being a hydrogen atom and R⁸ being a methyl group.

4. An additive for food and beverage according to claim 1,
wherein the compound represented by the general formula (1) or a salt thereof is derived from Banaba leaves.

5. A beverage comprising an additive for food and beverage according to claim 1.

6. A beverage comprising an additive for food and beverage according to claim 2.

7. A beverage comprising an additive for food and beverage according to claim 3.

8. A beverage comprising an additive for food and beverage according to claim 4.

9. A food comprising an additive for food and beverage according to claim 1.

10. A food comprising an additive for food and beverage according to claim 2.

11. A food comprising an additive for food and beverage according to claim 3.

12. A food comprising an additive for food and beverage according to claim 4.

13. An additive for food and beverage comprising:
at least one compound or salt thereof selected from the group consisting of: maslinic acid represented by the general formula (1) in which R¹ is a hydrogen atom, R² is a hydroxyl group, R³ is a hydroxyl group, R⁴ is a hydrogen atom, R⁵ is a carboxyl group, R⁶ is a methyl group, R⁷ is a methyl group and R⁸ is a hydrogen atom; ursolic acid represented by the general formula (1) in which R¹ is a hydrogen atom, R² is a hydrogen atom, R³ is a hydroxyl group, R⁴ is a hydrogen atom, R⁵ is a carboxyl group, R⁶ is a methyl group, R⁷ is a hydrogen atom and R⁸ is a methyl group; and oleanolic acid represented by the general formula (1) in which R¹ is a hydrogen atom, R² is a hydrogen atom, R³ is a hydroxyl group, R⁴ is a hydrogen atom, R⁵ is a carboxyl group, R⁶ is a methyl group, R⁷ is a methyl group and R⁸ is a hydrogen atom; and
corosolic acid represented by the general formula (1) or a salt thereof in which R¹ is a hydrogen atom, R² is a hydroxyl group, R³ is a hydroxyl group, R⁴ is a hydrogen atom, R⁵ is a carboxyl group, R⁶ is a methyl group, R⁷ is a hydrogen atom and R⁸ is a methyl group.

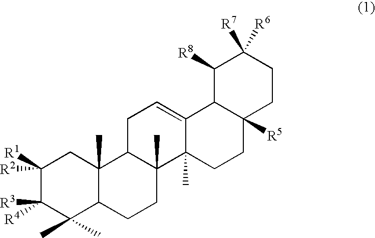

14. An additive for food and beverage according to claim 13 comprising:
at least one compound or salt thereof selected from the group consisting of 0.1 to 10 parts by weight of the maslinic acid, 0.1 to 10 parts by weight of the ursolic acid and 0.1 to 10 parts by weight of the oleanolic acid; and
100 parts by weight of the corosolic acid or a salt thereof.

15. An additive for food and beverage according to claim 13,
wherein the compound represented by the general formula (1) or a salt thereof is derived from Banaba leaves.

16. A beverage comprising an additive for food and beverage according to claim 13.

17. A beverage comprising an additive for food and beverage according to claim 14.

18. A beverage comprising an additive for food and beverage according to claim 15.

19. A food comprising an additive for food and beverage according to claim 13.

20. A food comprising an additive for food and beverage according to claim 14.

21. A food comprising an additive for food and beverage according to claim 15.

22. A pharmaceutical composition for treating or preventing diabetes or hyperglycemia comprising a compound represented by the general formula (1) or a salt thereof as an active ingredient: 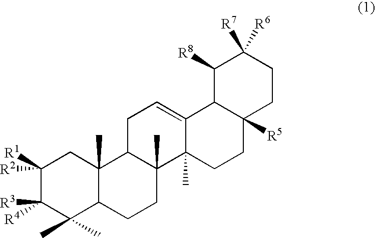
wherein
R¹, R², R³ and R⁴ independently represent a glycoside group consisting of 1 to 10 monosaccharide units, a hydroxyl group or a hydrogen atom,
R⁵ represents a carboxyl group, a methyl group, a methylol group, a formyl group or a group represented by —COR¹⁰,
R⁶, R⁷ and R⁸ independently represent a methyl group or a hydrogen atom,
wherein R¹⁰ is a glycoside group consisting of 1 to 10 monosaccharide units, and two of R⁶, R⁷ and R⁸ are each methyl group.

23. An pharmaceutical composition according to claim 22,
wherein the combination of R¹, R², R³, R⁴, R⁵, R⁶, R⁷ and R⁸ in the general formula (1) is:
R¹ being a hydrogen atom, R² being a hydroxyl group, R³ being a hydroxyl group, R⁴ being a hydrogen atom, R⁵ being a carboxyl group, R⁶ being a methyl group, R⁷ being a hydrogen atom and R⁸ being a methyl group;
R¹ being a hydrogen atom, R² being a hydroxyl group, R³ being a hydroxyl group, R⁴ being a hydrogen atom, R⁵ being a carboxyl group, R⁶ being a methyl group, R⁷ being a methyl group and R⁸ being a hydrogen atom;
R¹ being a hydrogen atom, R² being a hydrogen atom, R³ being a hydroxyl group, R⁴ being a hydrogen atom, R⁵ being a carboxyl group, R⁶ being a methyl group, R⁷ being a hydrogen atom and R⁸ being a methyl group; or
R¹ being a hydrogen atom, R² being a hydrogen atom, R³ being a hydroxyl group, R⁴ being a hydrogen atom, R⁵ being a carboxyl group, R⁶ being a methyl group, R⁷ being a methyl group and R⁸ being a hydrogen atom.

24. A pharmaceutical composition according to claim 22,
wherein the combination of R¹, R², R³, R⁴, R⁵, R⁶, R⁷ and R⁸ in the general formula (1) is R¹ being a hydrogen atom, R² being a hydroxyl group, R³ being a hydroxyl group, R⁴ being a hydrogen atom, R⁵ being a carboxyl group, R⁶ being a methyl group, R⁷ being a hydrogen atom and R⁸ being a methyl group.

25. A pharmaceutical composition according to claim 22,
wherein the pharmaceutical composition is an oral administration type.

26. A pharmaceutical composition according to claim 22,
wherein the compound represented by the general formula (1) or a salt thereof is derived from Banaba leaves.

27. A pharmaceutical composition for treating or preventing diabetes or hyperglycemia comprising:
at least one compound or salt thereof selected from the group consisting of: maslinic acid represented by the general formula (1) in which R¹ is a hydrogen atom, R² is a hydroxyl group, R³ is a hydroxyl group, R⁴ is a hydrogen atom, R⁵ is a carboxyl group, R⁶ is a methyl group, R⁷ is a methyl group and R⁸ is a hydrogen atom; ursolic acid represented by the general formula (1) in which R¹ is a hydrogen atom, R² is a hydrogen atom, R³ is a hydroxyl group, R⁴ is a hydrogen atom, R⁵ is a carboxyl group, R⁶ is a methyl group, R⁷ is a hydrogen atom and R⁸ is a methyl group; and oleanolic acid represented by the general formula (1) in which R¹ is a hydrogen atom, R² is a hydrogen atom, R³ is a hydroxyl group, R⁴ is a hydrogen atom, R⁵ is a carboxyl group, R⁶ is a methyl group, R⁷ is a methyl group and R⁸ is a hydrogen atom; and
corosolic acid represented by the general formula (1) or a salt thereof in which R¹ is a hydrogen atom, R² is a hydroxyl group, R³ is a hydroxyl group, R⁴ is a hydrogen atom, R⁵ is a carboxyl group, R⁶ is a methyl group, R⁷ is a hydrogen atom and R⁸ is a methyl group.

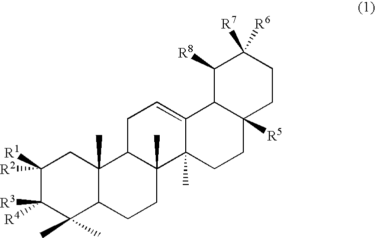

28. A pharmaceutical composition according to claim 27 comprising:
at least one compound or salt thereof selected from the group consisting of 0.1 to 10 parts by weight of the maslinic acid, 0.1 to 10 parts by weight of the ursolic acid and 0.1 to 10 parts by weight of the oleanolic acid; and
100 parts by weight of the corosolic acid or a salt thereof.

29. A pharmaceutical composition according to claim 27,
wherein the pharmaceutical composition is an oral administration type.

30. A pharmaceutical composition according to claim 27,
wherein the compound represented by the general formula (1) or a salt thereof is derived from Banaba leaves.

31. A GLUT4 translocator for translocating glucose transporter type 4 (GLUT4) onto cell membrane comprising a compound represented by the general formula (1) or a salt thereof as an active ingredient: 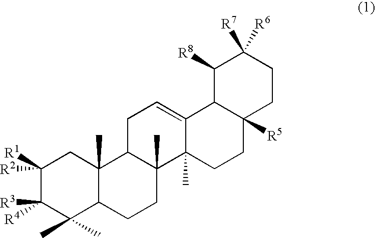
wherein
R¹, R², R³ and R⁴ independently represent a glycoside group consisting of 1 to 10 monosaccharide units, a hydroxyl group or a hydrogen atom,
R⁵ represents a carboxyl group, a methyl group, a methylol group, a formyl group or a group represented by —COR¹⁰,
R⁶, R⁷ and R⁸ independently represent a methyl group or a hydrogen atom,
wherein R¹⁰ is a glycoside group consisting of 1 to 10 monosaccharide units, and two of R⁶, R⁷ and R⁸ are each methyl group.

32. A GLUT4 translocator according to claim 31,
wherein the combination of R¹, R², R³, R⁴, R⁵, R⁶, R⁷ and R⁸ in the general formula (1) is:
R¹ being a hydrogen atom, R² being a hydroxyl group, R³ being a hydroxyl group, R⁴ being a hydrogen atom, R⁵ being a carboxyl group, R⁶ being a methyl group, R⁷ being a hydrogen atom and R⁸ being a methyl group;
R¹ being a hydrogen atom, R² being a hydroxyl group, R³ being a hydroxyl group, R⁴ being a hydrogen atom, R⁵ being a carboxyl group, R⁶ being a methyl group, R⁷ being a methyl group and R⁸ being a hydrogen atom;
R¹ being a hydrogen atom, R² being a hydrogen atom, R³ being a hydroxyl group, R⁴ being a hydrogen atom, R⁵ being a carboxyl group, R⁶ being a methyl group, R⁷ being a hydrogen atom and R⁸ being a methyl group; or
R¹ being a hydrogen atom, R² being a hydrogen atom, R³ being a hydroxyl group, R⁴ being a hydrogen atom, R⁵ being a carboxyl group, R⁶ being a methyl group, R⁷ being a methyl group and R⁸ being a hydrogen atom.

33. A GLUT4 translocator according to claim 31,
wherein the combination of R¹, R², R³, R⁴, R⁵, R⁶, R⁷ and R⁸ in the general formula (1) is R¹ being a hydrogen atom, R² being a hydroxyl group, R³ being a hydroxyl group, R⁴ being a hydrogen atom, R⁵ being a carboxyl group, R⁶ being a methyl group, R⁷ being a hydrogen atom and R⁸ being a methyl group.

34. A GLUT4 translocator according to claim 31,
wherein the compound represented by the general formula (1) or a salt thereof is derived from Banaba leaves.

35. A GLUT4 translocator for translocating glucose transporter type 4 (GLUT4) onto cell membrane comprising:
at least one compound or salt thereof selected from the group consisting of: maslinic acid represented by the general formula (1) in which R¹ is a hydrogen atom, R² is a hydroxyl group, R³ is a hydroxyl group, R⁴ is a hydrogen atom, R⁵ is a carboxyl group, R⁶ is a methyl group, R⁷ is a methyl group and R⁸ is a hydrogen atom; ursolic acid represented by the general formula (1) in which R¹ is a hydrogen atom, R² is a hydrogen atom, R³ is a hydroxyl group, R⁴ is a hydrogen atom, R⁵ is a carboxyl group, R⁶ is a methyl group, R⁷ is a hydrogen atom and R⁸ is a methyl group; and oleanolic acid represented by the general formula (1) in which R¹is a hydrogen atom, R² is a hydrogen atom, R³ is a hydroxyl group, R⁴ is a hydrogen atom, R⁵ is a carboxyl group, R⁶ is a methyl group, R⁷ is a methyl group and R⁸ is a hydrogen atom; and
corosolic acid represented by the general formula (1) or a salt thereof in which R¹ is a hydrogen atom, R² is a hydroxyl group, R³ is a hydroxyl group, R⁴ is a hydrogen atom, R⁵ is a carboxyl group, R⁶ is a methyl group, R⁷ is a hydrogen atom and R⁸ is a methyl group.

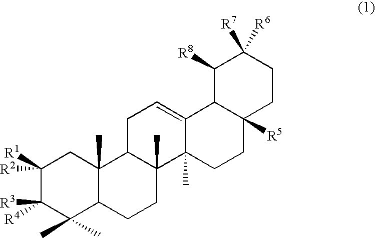

36. The GLUT4 translocator according to claim 35 comprising:
at least one compound or salt thereof selected from the group consisting of 0.1 to 10 parts by weight of the maslinic acid, 0.1 to 10 parts by weight of the ursolic acid and 0.1 to 10 parts by weight of the oleanolic acid; and
100 parts by weight of the corosolic acid or a salt thereof.

37. A GLUT4 translocator according to claim 35,
wherein the compound represented by the general formula (1) or a salt thereof is derived from Banaba leaves.

38. A method for translocating glucose transporter type 4 (GLUT4) onto cell membrane comprising a step of orally administrating a composition containing a compound represented by the general formula (1) or a salt thereof before meal: 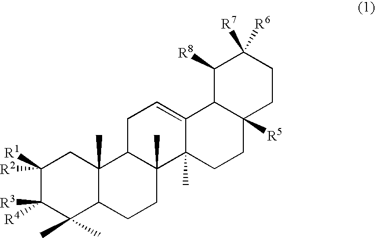
wherein
R¹, R², R³ and R⁴ independently represent a glycoside group consisting of 1 to 10 monosaccharide units, a hydroxyl group or a hydrogen atom,
R⁵ represents a carboxyl group, a methyl group, a methylol group, a formyl group or a group represented by —COR¹⁰,
R⁶, R⁷ and R⁸ independently represent a methyl group or a hydrogen atom,
wherein R¹⁰ is a glycoside group consisting of 1 to 10 monosaccharide units, and two of R⁶, R⁷ and R⁸ are each methyl group.

39. A method according to claim 38,
wherein the combination of R¹, R², R³, R⁴, R⁵, R⁶, R⁷ and R⁸ in the general formula (1) is:
R¹ being a hydrogen atom, R² being a hydroxyl group, R³ being a hydroxyl group, R⁴ being a hydrogen atom, R⁵ being a carboxyl group, R⁶ being a methyl group, R⁷ being a hydrogen atom and R⁸ being a methyl group;
R¹ being a hydrogen atom, R² being a hydroxyl group, R³ being a hydroxyl group, R⁴ being a hydrogen atom, R⁵ being a carboxyl group, R⁶ being a methyl group, R⁷ being a methyl group and R⁸ being a hydrogen atom;
R¹ being a hydrogen atom, R² being a hydrogen atom, R³ being a hydroxyl group, R⁴ being a hydrogen atom, R⁵ being a carboxyl group, R⁶ being a methyl group, R⁷ being a hydrogen atom, and R⁸ being a methyl group; or
R¹ being a hydrogen atom, R² being a hydrogen atom, R³ being a hydroxyl group, R⁴ being a hydrogen atom, R⁵ being a carboxyl group, R⁶ being a methyl group, R⁷ being a methyl group and R⁸ being a hydrogen atom.

40. A method according to claim 38,
wherein the combination of R¹, R², R³, R⁴, R⁵, R⁶, R⁷ and R⁸ in the general formula (1) is R¹ being a hydrogen atom, R² being a hydroxyl group, R³ being a hydroxyl group, R⁴ being a hydrogen atom, R⁵ being a carboxyl group, R⁶ being a methyl group, R⁷ being a hydrogen atom and R⁸ being a methyl group.

41. A method according to claim 38,
wherein the compound represented by the general formula (1) or a salt thereof is derived from Banaba leaves.

42. A method for translocating glucose transporter type 4 (GLUT4) onto cell membrane comprising a step of orally administrating before meal a composition containing:
at least one compound or salt thereof selected from the group consisting of: maslinic acid represented by the general formula (1) in which R¹ is a hydrogen atom, R² is a hydroxyl group, R³ is a hydroxyl group, R⁴ is a hydrogen atom, R⁵ is a carboxyl group, R⁶ is a methyl group, R⁷ is a methyl group and R⁸ is a hydrogen atom; ursolic acid represented by the general formula (1) in which R¹ is a hydrogen atom, R² is a hydrogen atom, R³ is a hydroxyl group, R⁴ is a hydrogen atom, R⁵ is a carboxyl group, R⁶ is a methyl group, R⁷ is a hydrogen atom and R⁸ is a methyl group; and oleanolic acid represented by the general formula (1) in which R¹ is a hydrogen atom, R² is a hydrogen atom, R³ is a hydroxyl group, R⁴ is a hydrogen atom, R⁵ is a carboxyl group, R⁶ is a methyl group, R⁷ is a methyl group and R⁸ is a hydrogen atom; and
corosolic acid represented by the general formula (1) or a salt thereof in which R¹ is a hydrogen atom, R² is a hydroxyl group, R³ is a hydroxyl group, R⁴ is a hydrogen atom, R⁵ is a carboxyl group, R⁶ is a methyl group, R⁷ is a hydrogen atom and R⁸ is a methyl group.

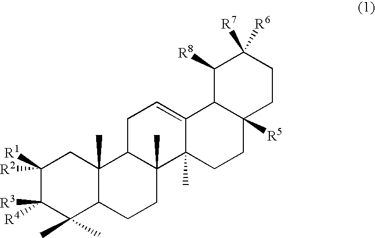

43. A method according to claim 42 comprising a step of orally administrating before meal a composition containing:
at least one compound or salt thereof selected from the group consisting of 0.1 to 10 parts by weight of the maslinic acid, 0.1 to 10 parts by weight of the ursolic acid and 0.1 to 10 parts by weight of the oleanolic acid; and
100 parts by weight of the corosolic acid or a salt thereof.

44. A method according to claim 42,
wherein the compound represented by the general formula (1) or a salt thereof is derived from Banaba leaves.
